(19) United States
(12) Patent Application Publication
(10) Pub. No.: US 2006/0241586 A1
(43) Pub. Date: Oct. 26, 2006

(54) Intra-abdominal medical device and associated method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/387,523

(22) Filed: Mar. 23, 2006

Related U.S. Application Data
(60) Provisional application No. 60/674,039, filed on Apr. 22, 2005.

(51) Int. Cl. A61B 18/14

(52) U.S. Cl. 606/45; 606/49

(57) ABSTRACT

A surgical method utilizes an instrument including elongate tube having a lumen, an elongate rod disposed in the lumen, and a screw thread disposed along at least a distal end portion of the rod. The method comprises inserting a distal end portion of the tube into an organ inside patient and thereafter rotating at least the distal end portion of the rod so that the screw thread draws material from the patient in a proximal direction along the lumen.

### CROSS-REFERENCE TO RELATED APPLICATION

This application claims the benefit of U.S. Provisional Patent Application No. 60/674,039 filed Apr. 22, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures, such as the difficulty in forming a fluid tight closure of the perforation formed in the wall of the hollow internal body organ. Certain intra-abdominal operations cannot be easily performed owing to the necessity of removing large chunks of organic or inorganic material (e.g., entire kidney, myoma from uterus). Some operations can require the simultaneous usage of many different instruments so that space along the selected pathways may be difficult to find.

### OBJECTS OF THE INVENTION

It is an object of the present invention to provide improvements on the afore-described surgical procedures.

It is another object of the present invention to provide a method and/or an associated device for facilitating the removal of large amounts of organic material from a patient, for instance, from the abdomen.

A further object of the present invention is to provide a method and/or an associated device for use in rigid laparoscopy and/or flexible trans-organ surgery.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical device comprises an elongate flexible tube having a lumen, an elongate flexible rod disposed in the lumen, and a screw thread disposed along at least a distal end portion of the rod. Preferably this surgical device further comprises a coupling component at a proximal end of the rod for rotating the rod about a longitudinal axis, whereby the screw draws matter along the lumen from a distal end of the tube to the proximal end thereof. This device is essentially a flexible or bendable Archimedes screw.

The device in accordance with the present invention can be used in endoscopic surgical procedures, for example, in trans-organ surgical operations as described in U.S. Pat. Nos. 5,297,536 and 5,458,131, to remove large organs masses such as a myoma from a uterus. Other operations could conceivably involve trans-section of the liver, the pancreas or a kidney.

The surgical device may additionally comprise an electrocautery electrode disposed at a distal end of the tube.

The rod is preferably formed at a distal end with a sharp tip. Additionally or alternatively, the screw thread is formed at a distal end of the rod with a sharp edge. These features facilitate the penetration and removal of hard masses such as a myoma.

A surgical method in accordance with the present invention utilizes an instrument including elongate tube having a lumen, an elongate rod disposed in the lumen, and a screw thread disposed along at least a distal end portion of the rod. The method comprises inserting a distal end portion of the tube into an organ inside the patient and thereafter rotating at least the distal end portion of the rod so that the screw thread draws material from the patient in a proximal direction along the lumen.

The distal end of the tube may be inserted into the organ multiple times to remove multiple tissue along multiple paths.

Where the tube and the rod are flexible, the inserting of the distal end portion of the tube includes moving the distal end portion of the tube along an at least partially curved path into the patient. As discussed in U.S. Pat. Nos. 5,297,536 and 5,458,131, the path may extend through a natural body opening such as the mouth, vagina orifice, or anus, through a hollow internal organ such as the stomach, vagina, or colon, through an artificial opening formed in a wall of that internal organ, and into the abdominal cavity. The hollow internal organ may alternatively be the urinary bladder.

The inserting of the distal end portion of the tube may alternatively include passing the distal end portion of the tube through the patient's abdominal wall into the patient's abdominal cavity. This procedure would be used during laparoscopic surgery.

Pursuant to another feature of the present invention, where the tube is provided at a distal end with an electrocautery electrode, the method further comprises conducting an electrical current through the electrode to coagulate blood vessels during the inserting of the distal end portion of the tube into the portion of the organ.

Pursuant to a further feature of the present invention, the method further comprises ablating the organ portion from another part of the organ prior to the inserting of the distal end portion of the tube into the organ portion.

According to another feature of the invention, the method further comprises inserting the ablated portion of the organ into a bag or pouch. In that event, the inserting of the distal end portion of the tube into the organ portion includes inserting the distal end portion of the tube into the bag or pouch.

### BRIEF DESCRIPTION OF THE DRAWING

FIG. 1 is a schematic longitudinal cross-sectional view of a surgical instrument in accordance with the present invention.

FIGS. 2A-2C are a series of schematic elevational views of a human uterus, showing successive steps in an procedure removing a myoma from the uterus.

FIGS. 3A-3D are a series of schematic elevational views of a human uterus, showing successive steps in an alternative procedure removing a myoma from the uterus.

FIG. 4 is a schematic perspective view of a powered morcellator in accordance with the present invention.

FIG. 5 is a schematic elevational view of a flexible Archimedes screw being used to draw water or other liquid from a reservoir or other body of fluid.

### DETAILED DESCRIPTION

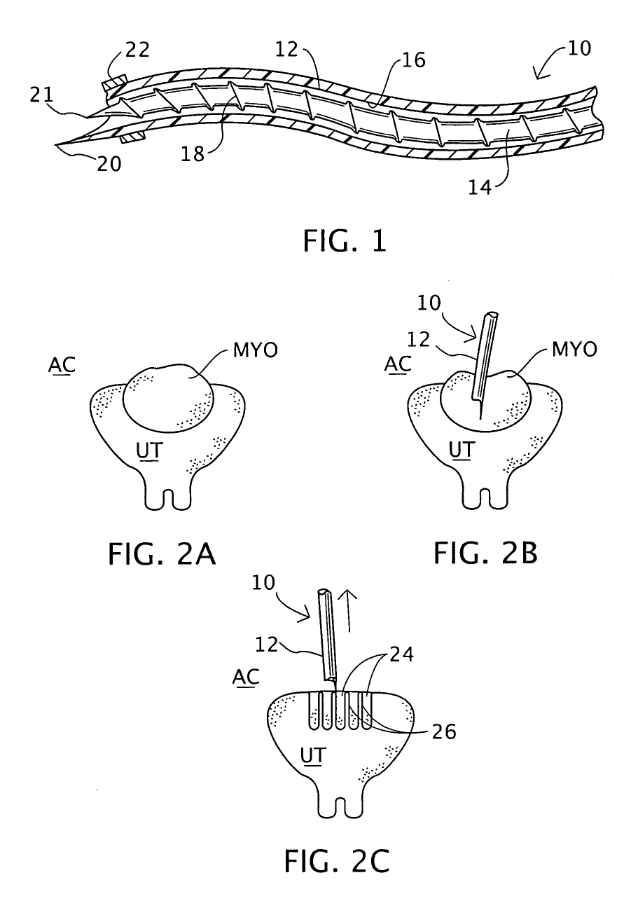

As depicted in FIG. 1, a surgical instrument 10 based on the classic Archimedes screw includes a tubular outer member 12, a rod member 14 disposed inside a lumen 16 of the tubular member, and a screw thread 18 attached at least to a distal end portion of the rod. Tubular member 12 is provided at a distal tip with a sharp point 20. Alternatively or additionally, rod member 14 is provided at a distal end with a sharp point 21. Also, screw thread 18 may be formed along a distal end portion with a sharp cutting edge (not separately designated). In at least some applications, for example in trans-organ surgery as described in U.S. Pat. Nos. 5,297,536 and 5,458,131, tubular member 12 and rod member 14 are flexible to enabling a flexing or bending of the instrument as it is being inserted along a curvilinear path through a patient's body.

Pursuant to U.S. Pat. Nos. 5,297,536 and 5,458,131, the path may extend through a natural body opening such as the mouth, vagina orifice, or anus, through a hollow internal organ such as the stomach, vagina, or colon, through an artificial opening formed in a wall of that internal organ, and into the abdominal cavity (none shown). The hollow internal organ may alternatively be the urinary bladder.

As depicted in FIGS. 2A-2C, the distal end portion of instrument 10, including tubular outer member 12, is inserted into a patient's abdominal cavity AC and into a myoma MYO in a uterus UT. This insertion process, as well as the subsequent myoma removal, may be effectuated in the course of rigid laparoscopy or trans-organ surgery pursuant to U.S. Pat. Nos. 5,297,536 and 5,458,131. During the insertion of a distal end portion of instrument 10, including tubular member 12, rod member 14, and screw thread 18, into myoma MYO, rod member 14 is rotated about its longitudinal axis so that screw thread 18 morcellates the myoma tissues and pulls the comminuted material in a proximal direction along lumen 16. Also during insertion of a distal end portion of instrument 10 into myoma MYO, an electrical current is optionally generated in an electrocautery electrode 22 (FIG. 1) provided at the distal end of tubular member 12, thereby cauterizing tissues along the insertion path. Electrode 22 may take the form of a ring surrounding tubular member 12.

As depicted in FIGS. 2A-2C, the above-described procedure may be repeated multiple times to create multiple bores 24 in myoma MYO. Remaining filaments of tissue 26 may be morcellated and extracted by the same Archimedean process. Alternatively, or additionally, a cutting blade (not shown) may be inserted into bores 24 for severing filaments 26 at their bases. The filaments may be removed one-by-one along the laparoscopic or trans-organ insertion path.

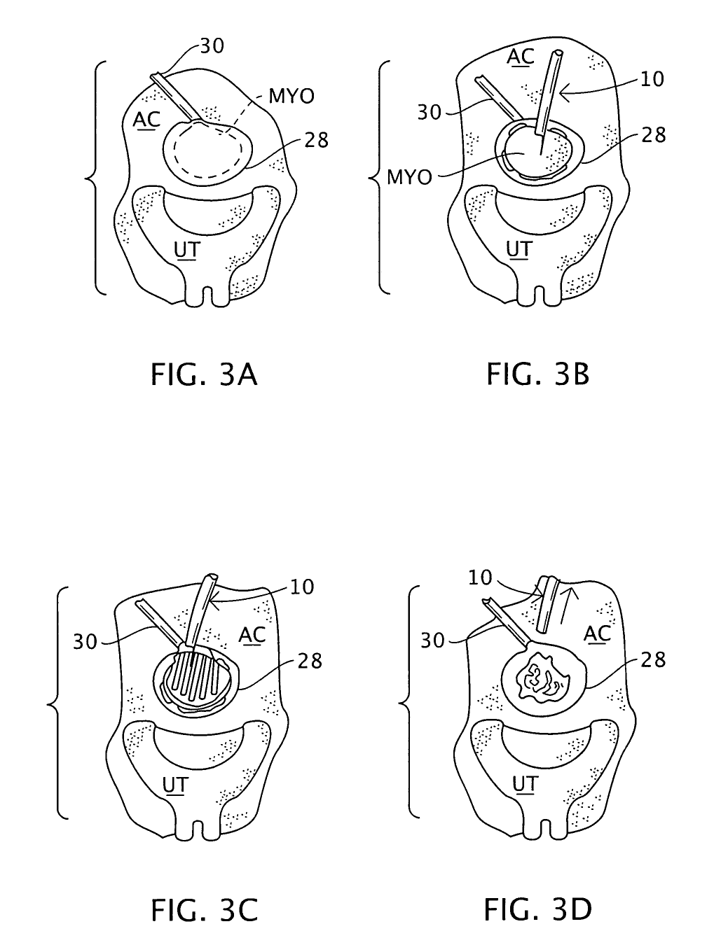

As illustrated in FIGS. 3A-3D, an alternative myoma removal procedure entails a laparascopic or trans-organ excision of the myoma MYO in bulk. The myoma is then placed in a pouch or bag 28 that is held in the abdominal cavity AC by a graspers 30 or dedicated rod. The distal end portion of instrument 10 is then inserted into the pouch or bag 28 to morcellate the excised mass of myoma MYO as discussed above with reference to FIG. 2.

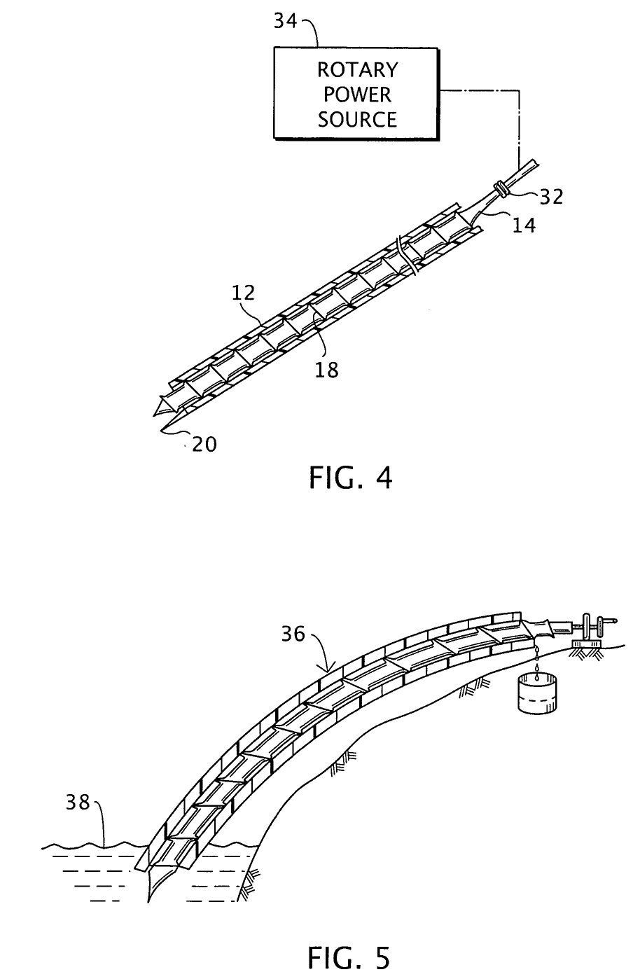

FIG. 4 depicts instrument 10 with rod member 14 connected at a proximal end via a coupling component 32 to a source 34 of rotary power. As indicated above, tubular member 12 and rod member 14 may be rigid for use particularly, but not necessarily exclusively, in laparascopic surgery or flexible for use particularly, but not necessarily exclusively, in trans-organ surgery.

FIG. 5 depicts a flexible Archimedes screw device 36 for use in drawing a liquid such as water or oil from a reservoir 38 or other body of fluid in an upward direction.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is understood that the drawings and descriptions herein are proffered by way of example to facilitate comprehension of the invention and should not be construed to limit the scope thereof.

What is claimed is:

1. A surgical device comprising:
an elongate flexible tube having a lumen;
an elongate flexible rod disposed in said lumen; and
a screw thread disposed along at least a distal end portion of said rod.

2. The surgical device defined in claim 1, further comprising means at a proximal end of said rod for rotating said rod about a longitudinal axis, so that said screw draws matter along said lumen from a distal end of said tube to said proximal end thereof.

3. The surgical device defined in claim 2 wherein said means for rotating includes a coupling component for connecting said rod to a source of rotary power.

4. The surgical device defined in claim 1, further comprising an electrocautery electrode disposed at a distal end of said tube.

5. The surgical device defined in claim 1 wherein at least one of said rod and said tube is formed at a distal end with a sharp tip.

6. The surgical device defined in claim 1 wherein said screw thread is formed at a distal end of said rod with a sharp edge.

7. A surgical method comprising:
providing an instrument including an elongate tube having a lumen, an elongate rod disposed in said lumen, and a screw thread disposed along at least a distal end portion of said rod;
inserting a distal end portion of said tube into a portion of an organ inside patient; and
thereafter rotating at least the distal end portion of said rod so that said screw thread draws material from said patient in a proximal direction along said lumen.

8. The method defined in claim 7 wherein said tube and said rod are flexible, the inserting of the distal end portion of said tube includes moving the distal end portion of said tube along an at least partially curved path into the patient.

9. The method defined in claim 8 wherein the inserting of the distal end portion of said tube includes passing the distal end portion of said tube into the patient through a natural body opening of the patient and through an aperture formed in a hollow internal organ of the patient.

10. The method defined in claim 7 wherein the inserting of the distal end portion of said tube includes passing the distal end portion of said tube through the patient's abdominal wall into the patient's abdominal cavity.

11. The method defined in claim 7 wherein said tube is provided at a distal end with an electrocautery electrode, further comprising conducting an electrical current through said electrode to coagulate blood vessels during the inserting the distal end portion of said tube into said portion of said organ.

12. The method defined in claim 7 wherein said organ is a uterus and said material constitutes myoma tissue.

13. The method defined in claim 7, further comprising ablating said portion of said organ from another part of said organ prior to the inserting of said distal end portion of said tube into said portion of said organ.

14. The method defined in claim 13, further comprising inserting the ablated portion of said organ into a bag or pouch, the inserting of said distal end portion of said tube into said portion of said organ including inserting said distal end portion of said tube into said bag or pouch.

15. A surgical device comprising an Archimedes screw having an outer tubular member and an internal screw member, said tubular member being provided at a distal end with an electrocautery electrode.

16. The surgical device defined in claim 15, wherein said tubular member and said screw member are at least partially flexible.
